(19) United States
(12) Patent Application Publication
(10) Pub. No.: US 2015/0044147 A1
(43) Pub. Date: Feb. 12, 2015

(54) COMPOSITION FOR MANAGEMENT OF PERIODONTAL DISEASE

(71) Applicants: KING ABDULLAH INTERNATIONAL MEDICAL RESEARCH CENTER, Riyadh (SA); KING SAUD BIN ABDULAZIZ UNIVERSITY FOR HEALTH SCIENCES, Riyadh (SA); NATIONAL GUARD HEALTH AFFAIRS, Riyadh (SA)

(72) Inventors: NOHA MOHAMED ZAKI RAYAD, TAIF (SA); IBRAHIM MAGHRABI, TAIF (SA); MOHAMED MOSTAFA HAFEZ MAHMOUD, TAIF (SA)

(21) Appl. No.: 14/521,438

(22) Filed: Oct. 22, 2014

Related U.S. Application Data
(63) Continuation-in-part of application No. 13/769,800, filed on Feb. 18, 2013, now abandoned.

(51) Int. Cl. A61K 9/16; A61K 31/24; A61K 47/36; A61K 47/10; A61K 31/5383; A61K 9/06

(52) U.S. Cl. CPC A61K 9/16; A61K 31/5383; A61K 9/06; A61K 47/36; A61K 47/10; A61K 9/1652; A61K 9/1647; A61K 31/24
USPC 424/52; 424/54

(57) ABSTRACT

The composition for management of periodontal diseases includes a gel matrix having a polymer system and a plurality of microspheres dispersed in the polymer system. The polymer system contains about one-half a dose of medicament, while the microspheres contain the remainder. Upon administration of the composition into the periodontal cavity, the medicament in the polymer system provides an initial therapeutic benefit, while the remainder of the medication is released over time via degradation of the microspheres. This biphasic pattern of medicament delivery provides increased efficacy of the medicament through sustained delivery of the same.

### CROSS-REFERENCE TO RELATED APPLICATION

This application is a continuation-in-part of U.S. patent application Ser. No. 13/769,800, filed Feb. 18, 2013, which is incorporated by reference herein.

### BACKGROUND OF THE INVENTION

1. Field of the Invention

The present invention relates to periodontal disease treatments, and particularly to a composition for management of periodontal diseases that provides maximal effective delivery of medicament into the periodontal pocket.

2. Description of the Related Art

Periodontal disease is an infection caused by bacteria in the biofilm or dental plaque that forms on oral surfaces. The disease causes deterioration of the teeth and gums in the oral cavity and typically manifests as lesions in various states of progression. Usually, the disease begins as gingivitis, an inflammation of the gums, which can lead to periodontitis, a condition in which the patient exhibits progressive loss of the alveolar bone around the teeth. Left untreated, the teeth will loosen and the patient will eventually lose the teeth.

The oral cavity is home to a host of bacteria, at least 500 or so identified bacterium, and the body is in constant struggle combating these bacteria. In general, waste products from these bacteria cause destruction of tissue and halitosis. Due to the complex etiology of these bacteria, it has been difficult to identify a particular pathogen for periodontal disease. However, recent advances in molecular biological techniques have enabled easier identification of periodontopathic bacteria.

Regular brushing and flossing are common measures that reduce risks of periodontal disease. However, many factors are involved with the onset of the disease. Studies have shown that while advanced age is a common factor, other factors, such as genetics, tobacco use, gender, and diabetes mellitus, are also found to be culpable.

Several treatments exist to counter periodontal diseases. One common form of treatment involves rinsing subgingival pockets with a solution of hydrogen peroxide, typically in concentrations of 1%-3%. The hydrogen peroxide acts as an antimicrobial agent. Another treatment involves an antibiotic, such as doxycycline, orally administered to the patient. A still further treatment involves injection of medication in the periodontal cavity. In the latter case, the efficacy of the medication is somewhat diminished due to hindered transmucosal delivery having an effect on absorption and the absorption rate.

In light of the above, it would be a benefit in the art of periodontal disease treatment to provide a composition that insures effective delivery of drugs to counter the effects of periodontal disease. Thus, a composition for management of periodontal diseases solving the aforementioned problems is desired.

### SUMMARY OF THE INVENTION

The composition for management of periodontal diseases includes a polymer system forming a gel matrix, and a plurality of microspheres dispersed in the polymer system. The polymer system contains about one-half the dose of medicament, while the microspheres contain the remainder. Upon administration of the composition into the periodontal cavity, the medicament in the polymer system provides an initial therapeutic benefit, while the remainder of the medication is released over time via degradation of the microspheres. This biphasic pattern of medicament delivery provides increased efficacy of the medicament through sustained delivery of the same.

These and other features of the present invention will become readily apparent upon further review of the following specification and drawings.

### BRIEF DESCRIPTION OF THE DRAWINGS

FIG. 1 is an environmental, perspective view of a composition for management of periodontal diseases being administered to a patient.

FIG. 2 is a chart comparing the antibacterial effect of the composition for management of periodontal diseases compared to a conventional solution over time.

FIG. 3 is a chart comparing the probing depth (PD) and clinical attachment level (CAL) reductions in patients treated with the composition for management of periodontal diseases and the conventional solution.

FIG. 4 is a diagram of a syringeability-measuring device.

FIG. 5 is a graph showing rheological changes of the chitosan/P188/P407 polymer system and the poloxamer-only polymer system.

FIG. 6 is a graph showing shear stress of formulations B, C, and E (from Table 1).

FIG. 7 is a graph showing the thixotropic pseudoplastic rheological behavior of in-situ gel including a poloxamer-only polymer system, of formula C (from Table 1), and of chitosan-only polymer system.

FIG. 8 is graph showing the inhibitory effect of chitosan-only polymer systems, poloxamer-only polymer systems (P188/P407), and chitosan/P188/P407 polymer systems on bacterial adherence to non-living surfaces.

FIG. 9 is a graph showing the inhibitory effect of chitosan-only polymer systems, poloxamer-only polymer systems (P188/P407), and chitosan/P188/P407 polymer systems on bacterial adherence to mammalian cells.

FIG. 10 is a graph showing the biofilm targeting effect of chitosan/P188/P407 polymer systems, poloxamer-only polymer systems, and chitosan-only polymer systems.

FIG. 11 is a graph showing minimum inhibitory concentration (MIC) of ofloxacin when ofloxacin is used with chitosan/P188/P407 polymer systems and the MIC of ofloxacin when ofloxacin is used in solution.

FIG. 12 is a graph showing the effect of formulations including chitosan/P188/P407 polymer systems on virulence factors (proteolytic activity) of the periodontal pathogen P. gingivalis.

Similar reference characters denote corresponding features consistently throughout the attached drawings.

### DETAILED DESCRIPTION OF THE PREFERRED EMBODIMENTS

The composition for management of periodontal diseases provides sustained therapeutic levels of medication delivery for treating periodontal disease. The composition includes a polymer system forming a gel matrix containing about one-half of a drug or medicament dose, and a plurality of microspheres dispersed in the polymer system, the microspheres containing the remainder of the prescribed drug dose.

The polymer system is configured to deliver rapid therapeutic levels of the drug into the gingival crevicular fluid (GCF). The polymer system can include chitosan provided in about 0.5-5% by weight concentration, and/or poloxamer members at about 16-30% by weight concentration alone or in a mixture of the above. Chitosan has proven to exhibit antibiofilm and antibacterial effect against periodontopathic bacteria, especially Parphyromonas gingivalis. Poloxamer has also been shown to demonstrate antadherence effect against bacteria. Hence, the poloxamer can counteract plaque formation.

The microspheres contain the remainder of the medicament and provide a time-release mechanism for delivering the rest of the dose over a predetermined period. This facilitates a controlled and sustained release of the medicament, which greatly enhances efficacy of the treatment. The microspheres are constructed from ethylcellulose (EC), poly(lactide-co-glycolide) polymers (PLGA), polycaprolactone (PCL), and the like that exhibit high biocompatibility and biodegradation. Preferably, the microspheres have a particle size ranging from about 50-800 _{R}m. The drug to polymer ratio is preferably about 1:2 or 1:4.

The drug or medicament for the composition can be an antibiotic or a local anesthetic. An exemplary antibiotic may be ofloxacin at about 0.1-1% concentration by weight, An exemplary anesthetic may be mebeverine HCl at about 10-50% concentration by weight. Although mebeverine HCl is more commonly used as an antispasmodic, especially for colon spasms, it has been found that the medicament demonstrates successful local anesthetic effect.

As briefly mentioned above, it is preferable that the polymers used in preparation of the composition exhibit a high degree of biocompatibility and biodegradation. The former is self-explanatory, while the latter insures a proper rate of deterioration for delivering the medicament. The above characteristics insure the composition remains in the periodontal cavity and administers the prescribed amount of medicament in a biphasic pattern for longer-lasting clinical improvement, i.e., the polymer system delivers the first half for an initial therapeutic benefit and the microspheres deliver the rest over time to extend that benefit. Moreover, the rheological properties are readily adjustable to facilitate ease of injection via a syringe and filling of the periodontal cavity.

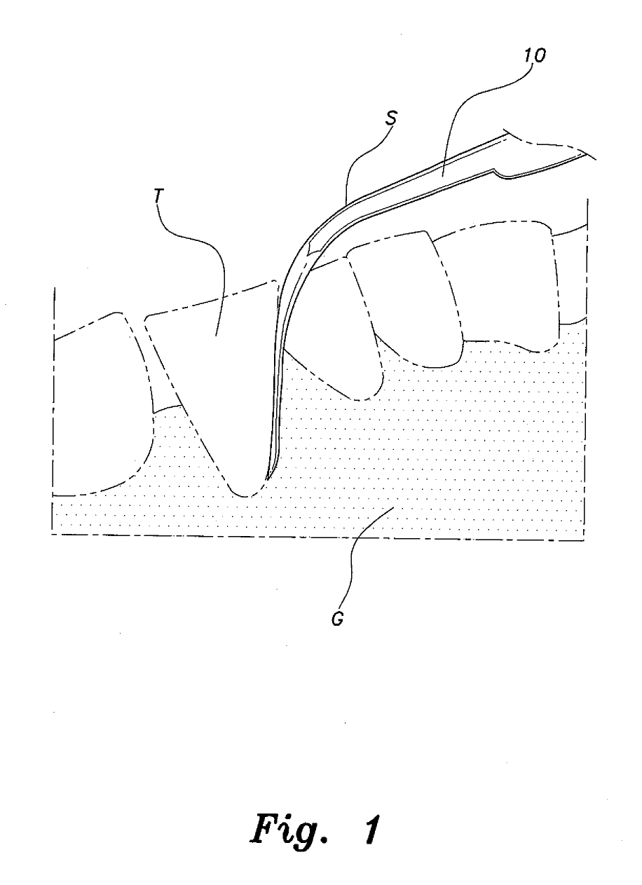

FIG. 1 shows an example of administering the composition 10. As shown, the dentist uses a syringe S filled with the composition 10 prepared in the manner described above. The syringe S is placed near the injection site between the teeth T and the gum G. The dentist injects the composition 10 into the periodontal cavity below the gum line.

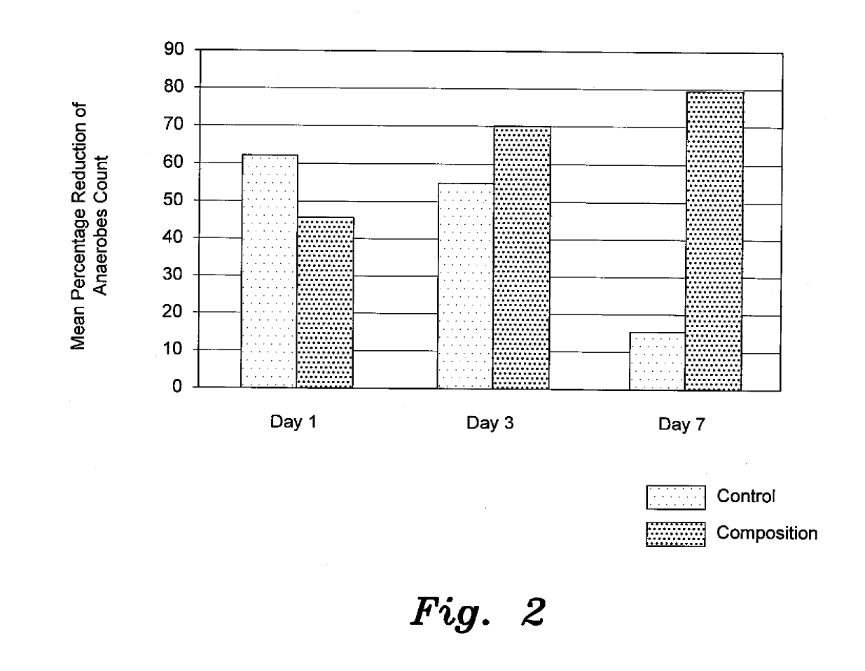

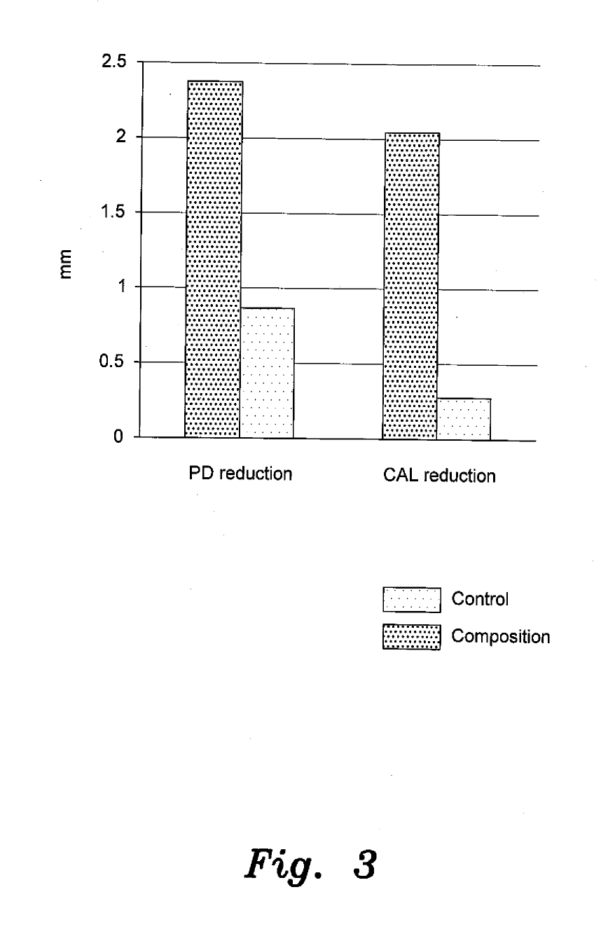

The above procedure has been used in tests, and the charts shown in FIGS. 2 and 3 demonstrate the effectiveness of the composition over injection of conventional medicament (indicated as “Control” in the drawings). FIG. 2 shows a comparison of the mean percentage reduction of anaerobes count between the composition 10 and the conventional modes of administering the medicament over a week, or seven days. Most bacteria responsible for periodontal disease are anaerobic. Thus, a greater percentage in anaerobe count reduction correlates to greater efficacy of the treatment. It can be seen from FIG. 2 that while the mean percentage of reduction of anaerobes was initially low, about 47% for the present composition compared to about 62% from the control, the therapeutic benefit of the composition was maintained and continuously increased throughout the week. At the end of the testing period, the composition exhibited about 78% anaerobes count reduction, while the control was substantially lower at about 15%. This suggests that while the initial or short term therapeutic benefit of the conventional medicament may be effective, the long term benefit of the composition far surpassed that of the conventional medicament.

FIG. 3 shows the comparison of probing depth (PD) and the clinical attachment level (CAL) between the composition and the control. PD is defined as the distance from the gingival margin to the bottom of the cavity or pocket, measured in millimeters (mm). PD serves as an indicator of the severity of the periodontal disease. CAL is defined as the distance from the cement-enamel junction (CEJ) to the bottom of the cavity, also measured in mm. CAL serves as the primary measure of the efficacy of the treatment. In both parameters, high reduction of either measurement indicates a greater degree of therapeutic treatment benefit. The results of FIG. 3 are derived from measurements taken from the beginning and end of the seven day period. It can be seen from FIG. 3 that the composition demonstrated a substantial reduction in both PD and CAL, compared to the control. The PD reduction was about 2.4 mm and 0.8 mm respectively. This suggests that the composition was about 300% more effective in healing the gum compared to the control. The CAL reduction was about 2.1 mm and 0.25 mm, respectively. This suggests that the efficacy of the composition was much greater than the efficacy from the control.

Thus, it can be seen that the composition 10 for managing periodontal disease provides improved therapeutic treatment of the disease. The gel matrix facilitates a biphasic delivery pattern for the medicament, which greatly increases the efficacy through long-term, sustained administration of the medicament.

Preferably, the polymer system includes both chitosan and poloxamer, e.g., poloxamer 407 and/or poloxamer 188. The polymer system can include for example, chitosan, poloxamer 407, and poloxamer 188 (“chitosan/P188/P407 polymer system”). The chitosan/P188/P407 polymer system can include, for example about 0.5% to about 1.5% by weight chitosan, about 20% to about 30% by weight poloxamer. Preferably, the chitosan/P188/P407 polymer system includes about 15% to about 20% by weight poloxamer 407 and about 5% to about 15% by weight poloxamer 188. The chitosan/P188/P407 polymer system can be used to deliver a medicament or active ingredient such as ofloxacin. Ofloxacin can be directly added to the gel (free in the in-situ gel) or can be in the form of microspheres. Exemplary formulations of a composition for management of periodontal diseases including the chitosan/P188/P407 polymer system and ofloxacin are provided below in Table 1.

TABLE 1
Exemplary formulations

| | Chitosan | | | Ofloxacin (% w/v) | |
|---|---|---|---|---|---|
| Code | (% w/v) | Poloxamer 407 | Poloxamer 188 | free in the in-situ gel | in microspheres |
| A | 0.5 | 18 | 5 | 0.05 | 0.05 |
| B | 0.5 | 18 | 10 | 0.05 | 0.05 |
| C | 1 | 18 | 10 | 0.05 | 0.05 |
| D | 1 | 18 | 15 | 0.05 | 0.05 |
| E | 1.5 | 20 | 5 | 0.05 | 0.05 |
| F | 1.5 | 20 | 10 | 0.05 | 0.05 |

The physicochemical properties, of the chitosan/P188/P407 polymer system are provided in Table 2 below.

TABLE 2
Properties of chitosan/P188/P407 polymer system

| | | | Mucoadhesive | Gelation | |
|---|---|---|---|---|---|
| | pH ± | Gelation Temperature | force | time | Syringeability* |
| Formula | SD | (° C. ± SD) | (dyne/cm² ± SD) | (min ± SD) | (Newton ± SD) |
| chitosan/P188/P407 | 6.91 ± | 34.1 ± 0.5 | 5.44 ± 0.56 | 2 ± 1 | 5.65 ± 2.00 |
| | 0.02 | | | | |

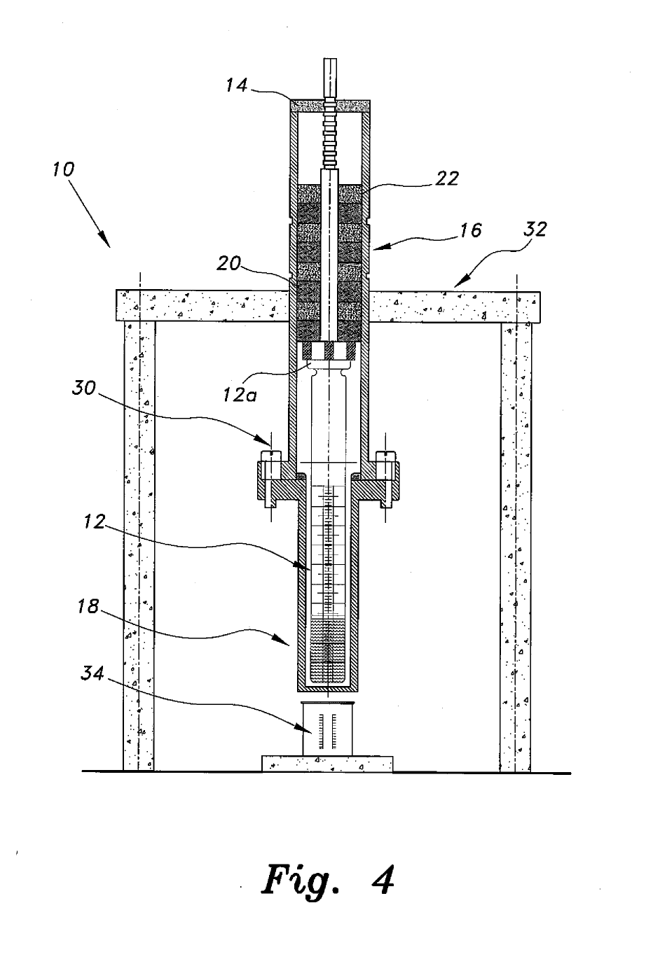

*measured using device shown in FIG. 4

The chitosan/P188/P407 polymer system demonstrated acceptable syringeability, i.e., could be effectively delivered by a syringe. As reflected in Table 2, the syringeability (Newton±SD) of the chitosan/P188/P407 ranges from about 3.65 to about 7.65. The pH of the chitosan/P188/P407 polymer system ranges from about 6.89 to about 6.93. The mucoadhesive force (dyne/cm′±SD) ranges from about 4.88 to about 6.0.

Syringeablity was measured with a syringeability-measuring device or system 10, as shown in FIG. 4. The syringeability-measuring system 10 includes a syringe 12 and a tubular syringe-holder 14 for retaining the syringe 12. The syringe holder 14 includes a top portion 16 and a bottom portion 18. A peripheral wall of the bottom portion 18 has a diameter slightly larger than the syringe 12 to receive and hold the syringe 12 therein. The top portion 16 includes a weight holding shell 20 that is configured to hold and receive weights 22 therein. The weight holding shell 20 is disposed adjacent a syringe plunger 12a. The top portion 16 and the bottom portion 18 are detachably connected with clamping screws 30. The syringe holder 14 is held in place by a support frame 32. A beaker 34 is disposed below the syringe 12. Syringeablity was measured by loading the syringe 12 in the bottom portion 18 with the polymer system. The top portion 16 was then attached to the bottom portion 18 using clamping screws 30. Weight holding shell 20 was then disposed in the top portion 16 and weights 22 were added one by one to determine the amount of pressure required to deliver the polymer system from the syringe into the beaker 34.

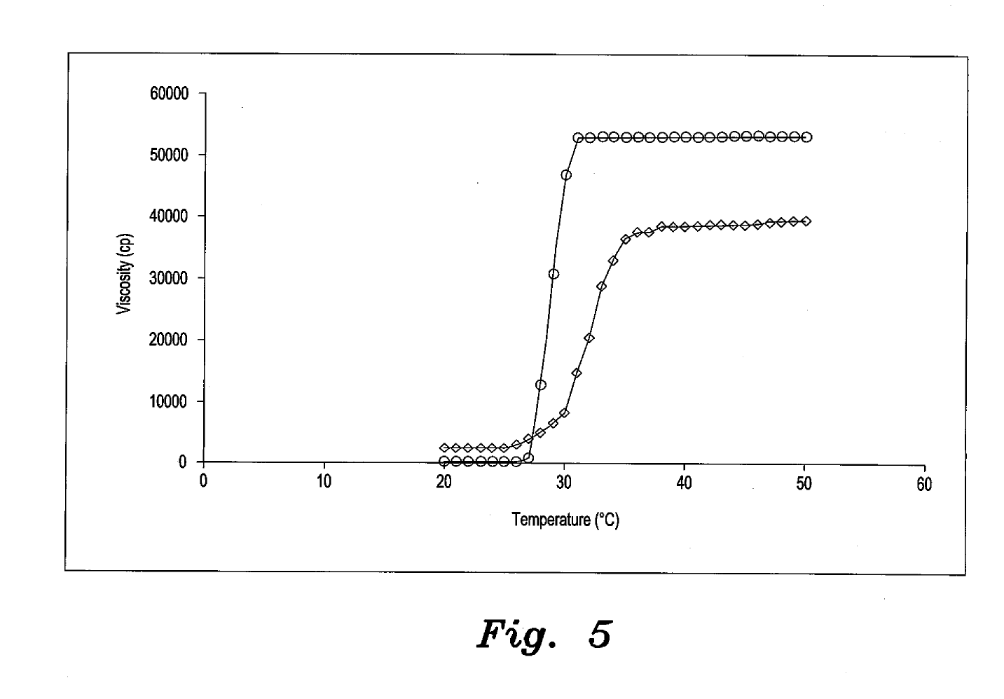

FIG. 5 reflects results of tests conducted to determine rheological changes of a polymer system including including the chitosan/P188/P407 polymer system, represented by diamonds in the graph and a polymer system that includes poloxamer but lacks chitosan (“poloxamer-only polymer system”, represented by circles in the graph). When rheological changes were measured, it was found that the chitosan/P188/P407 polymer system displayed sol-gel phase transition. As shown in FIG. 5, the chitosan/P188/P407 polymer system displayed a greater increase in viscosity as temperature was increased, compared to the poloxamer-only polymer system. This change in viscosity is characteristic of phase transition behavior. The sol-gel transition temperature of the chitosan/P188/P407 polymer system is about 33.6° C. to about 34.6° C.

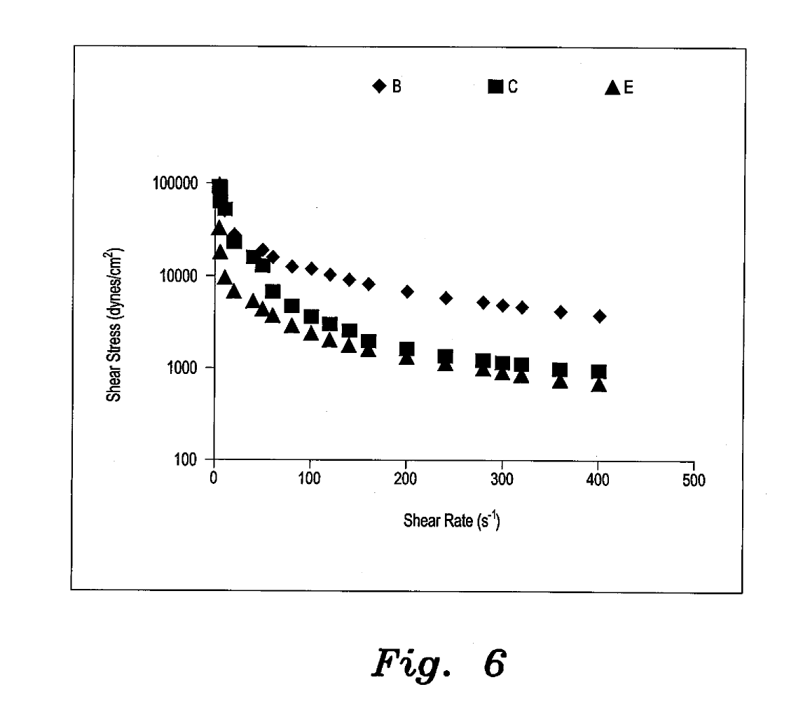

FIG. 6 reflects results of tests conducted to determine shear stress of formulations B, C, and E (from Table 1). FIG. 6 compares the shear stress of formulations B, C, and E, as a function of shear rate at 35° C.

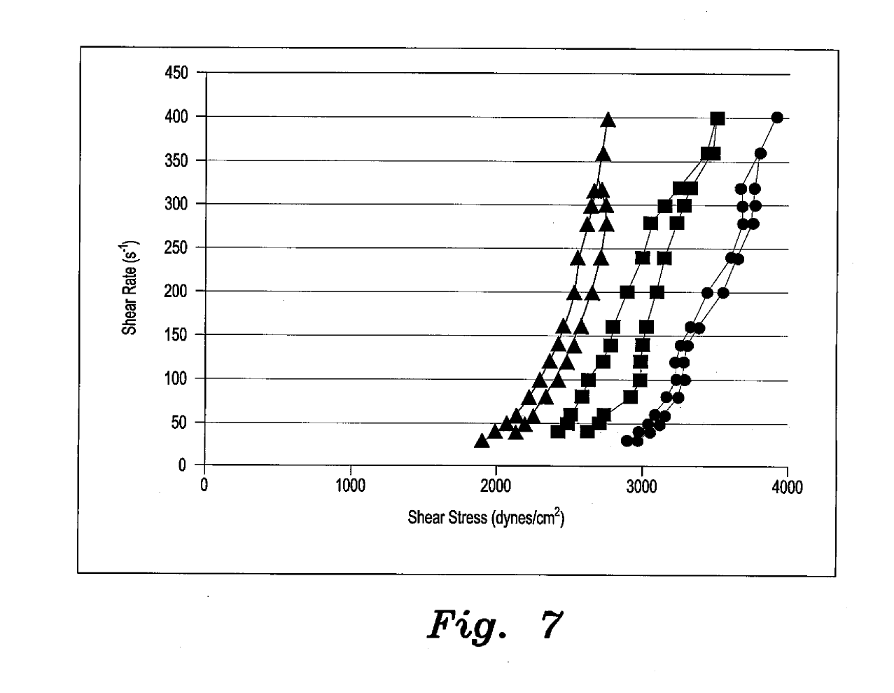

FIG. 7 reflects results of tests conducted to determine thixotropic pseudoplastic rheological behavior of in-situ gel including a poloxamer-only polymer system (P407/P188; 18/5), represented by triangles in the graph, of formulation C (from Table 1), represented by squares in the graph, and chitosan 0.1% gel including chitosan and lacking poloxamer (“chitosan-only polymer system”), represented by circles in the graph. As can be seen in FIG. 7, a higher thixotropic effect is observed with formulation C, as judged by the area under the hysteresis loop.

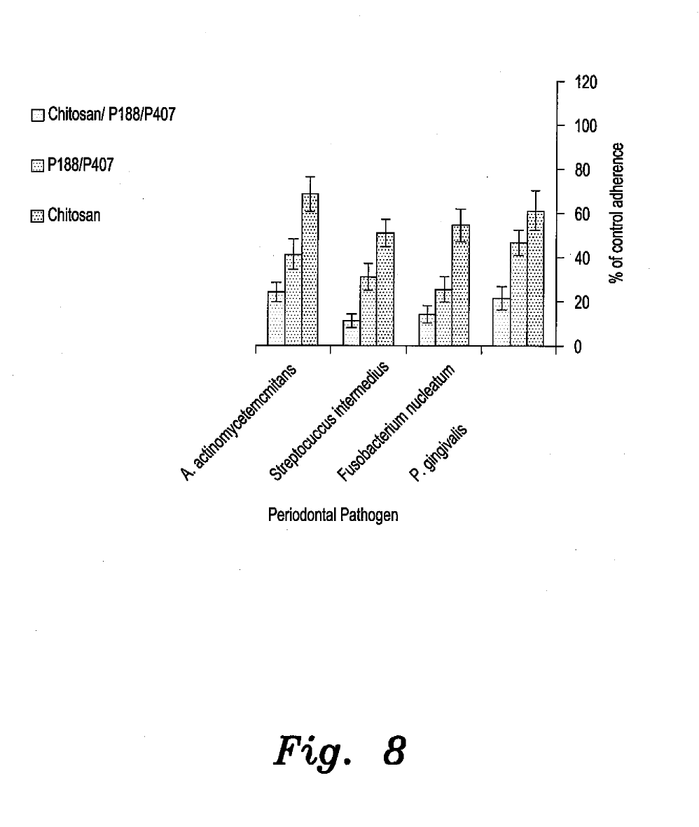

FIG. 8 reflects results of tests conducted to determine an inhibitory effect of a chitosan-only polymer system, a poloxamer-only polymer system (P188/P407), and the chitosan/P188/P407 polymer system on bacterial adherence to non-living surfaces. Bacterial adherence to non-living surfaces, e.g., teeth surface, is characteristic of dental infections. The chitosan/P188/P407 polymer system revealed a significant reduction in the adherence of periodontal pathogens (P. gingivalis, Fusobacterium nucleatum, Streptococcus intermedius, and A. actinomycetemcomitans) to non-living surfaces, a property which is particularly useful in hindering the onset of periodontitis. The chitosan/P188/P407 polymer system exhibited a pronounced synergistic anti-adherent effect, when compared to the poloxamer-only polymer system and the chitosan-only polymer system.

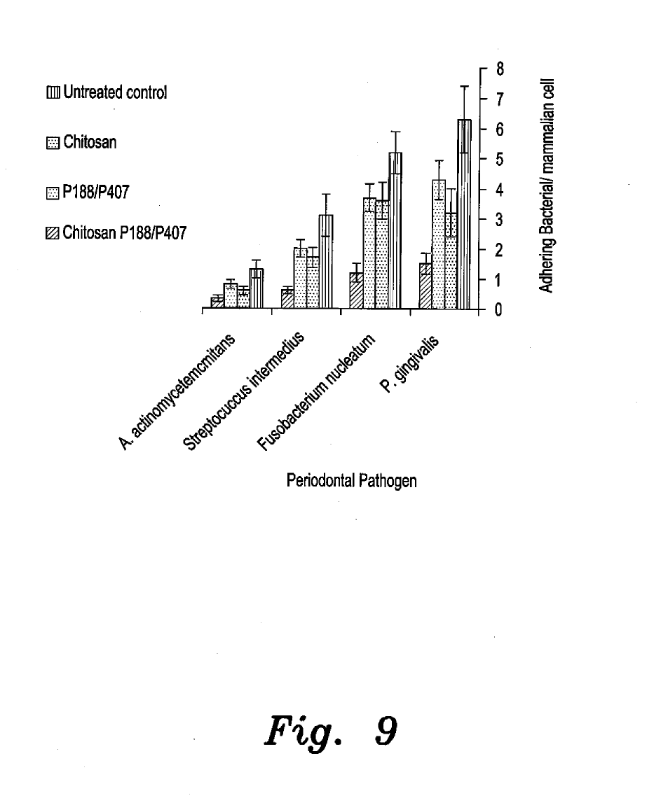

FIG. 9 reflects the results of tests conducted to determine an inhibitory effect of chitosan-only polymer systems, poloxamer-only polymer systems (P188/P407), and chitosan/P188/P407 polymer systems on bacterial adherence to mammalian cells. Bacterial adherence to mammalian cells is characteristic of infections. Without the ability to adhere to tissues, periodontal pathogens would be unable to establish or maintain the infection, form a hiofilm, or cause tissue damage. In this regard, chitosan/P188/P407 polymer systems were associated with significant reduction in the adherence of the periodontal pathogens (P. gingivalis, Fusobacterium nucleatum, Streptococcus intermedius, and A. actinomycetemcomitans) to Vero cells. A significantly more pronounced synergistic anti-adherent effect was observed with the chitosan/P188/P407 polymer system when compared with the chitosan-only and poloxamer-only polymer systems. Such anti-adherent effect was apparent in diminishing the development and progression of the periodontal disease as well as on the clinical outcome.

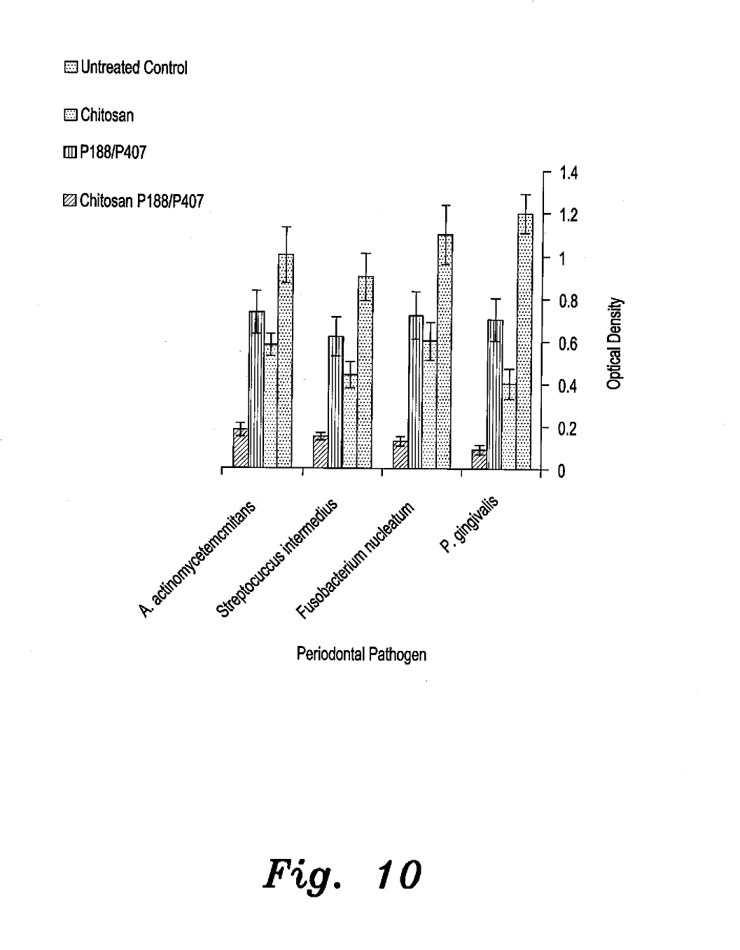

FIG. 10 reflects the results of tests conducted to determine biofilm targeting effect of chitosan/P188/P407 polymer systems, poloxamer-only polymer systems, and chitosan-only polymer systems. Chitosan-only polymer systems in the concentrations tested showed a significant inhibitory effect on biofilms of four main periodontal pathogens, namely, P. gingivalis, Fusobacterium nucleatum, Streptococcus intermedius, and A. actinomycetemcomitans. Similar but less pronounced anti-biofilm effect was shown by poloxamer-only polymer systems. Interestingly, chitosan/P188/P407 polymer systems revealed a significant synergistic anti-biofilm effect against the four tested pathogens with very little biofilm remaining. This finding indicates that the chitosan/P188/P407 polymer systems can be effective in preventing and counteracting the chronicity, immune evasion, and antimicrobial resistance of periodontitis.

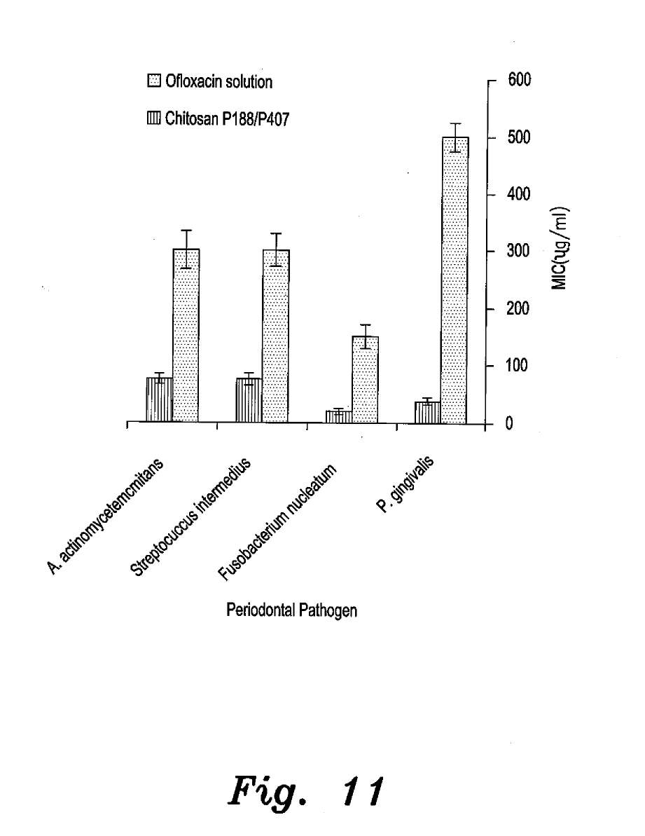

FIG. 11 reflects the results of testing done to determine the minimum inhibitory concentration (MIC) of ofloxacin when ofloxacin is used with chitosan/P188/P407 polymer systems and the MIC of ofloxacin when ofloxacin is used in solution. As shown in FIG. 11, formulations including the chitosan/P188/P407 polymer system showed significant capability to reduce the minimum inhibitory concentration (MIC) of ofloxacin with respect to four main periodontal pathogens (P. gingivalis, Fusobacterium nucleatum, Streptococcus intermedius, and A. actinomycetemcomitans). These results indicate that formulations including chitosan/P188/P407 polymer systems can offer the benefit of reducing antimicrobial resistance, the dose required to treat the periodontitis, and the possibility of adverse antibiotic effects.

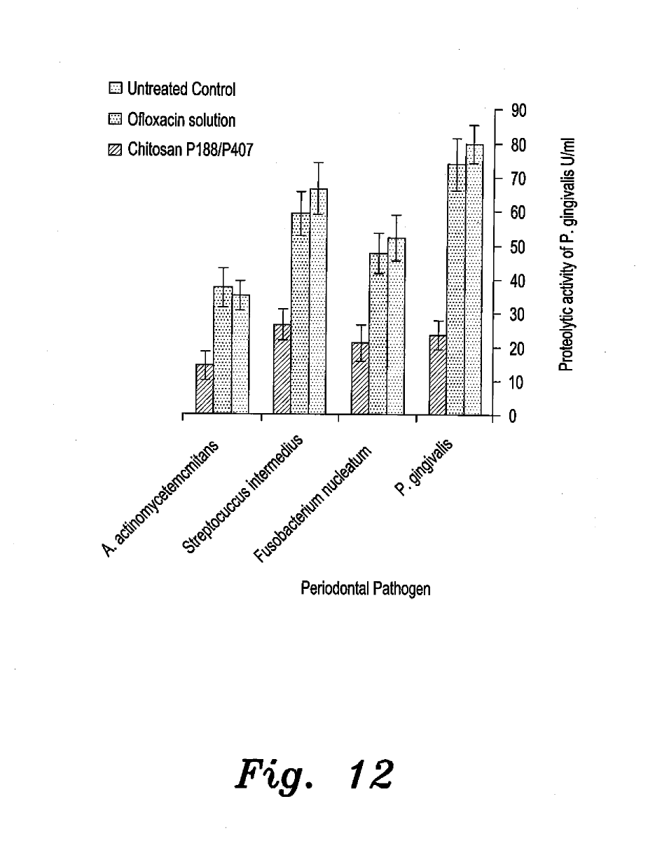

FIG. 12 reflects the results of testing done to determine the effect of formulations including chitosan/P188/P407 polymer systems on virulence factors (proteolytic activity) of the periodontal pathogen P. gingivalis. Proteolytic activity of the periodontal pathogens is a central virulence determinant for pathogenesis of periodontal disease. Such enzymatic activity enables destruction of host proteins and induction of inflammatory reactions that radically contribute to tissue damage in periodontitis. Indirectly, such tissue damage also facilitates the spread of infection. Interestingly, the chitosan/P188/P407 formulation showed a protease inhibiting potential against four main tested periodontal pathogens. Protease inhibition reduces the ability of periodontal pathogens to degrade host-derived proteins that cause tissue damage and inflammatory reactions. Consequently the severity and course of disease can be diminished.

It is to be understood that the present invention is not limited to the embodiments described above, but encompasses any and all embodiments within the scope of the following claims.

We claim:

1. A delivery system for management of periodontal diseases, comprising:
a polymer system forming a gel matrix adapted for carrying about one-half of a dose of a medicament for treating periodontal disease, the gel matrix providing rapid release of the medicament to achieve therapeutic levels, the gel matrix including about 0.5% to about 1.5% by weight chitosan and about 20% to about 30% by weight poloxamer members; and
a plurality of microspheres suspended and dispersed in the gel matrix, the microspheres containing the remainder of the dose, the microspheres providing gradual time release of the medicament to maintain the therapeutic levels of medicament for a sustained period of time.

2. The delivery system according to claim 1, wherein the poloxamer members include poloxamer 407 and poloxamer 188.

3. The delivery system according to claim 2, wherein said gel matrix comprises about 15% to about 20% by weight poloxamer 407 and about 5% to about 15% by weight poloxamer 188.

4. The delivery system according to claim 2, wherein said polymer system comprises about 18% by weight poloxamer 407 and about 10% by weight poloxamer 188.

5. The delivery system according to claim 2, wherein said polymer system comprises about 18% by weight poloxamer 407 and about 5% by weight poloxamer 188.

6. The delivery system according to claim 2, wherein said polymer system comprises said polymer system comprises about 20% by weight poloxamer 407 and about 10% by weight poloxamer 188.

7. The delivery system according to claim 2, wherein said said polymer system comprises about 20% by weight poloxamer 407 and about 5% by weight poloxamer 188.

8. The delivery system according to claim 1, wherein said microspheres are constructed from biodegradable and biocompatible polymers.

9. The delivery system according to claim 1, wherein said microspheres comprise ethylcellulose.

10. The delivery system according to claim 1, wherein said microspheres comprise a polymer selected from the group consisting of poly(lactide-co-glycolide) polymer and polycaprolactone polymer.

11. The delivery system according to claim 1, wherein the polymer system has a syringeability (Newton±SD) of from about 3.65 to about 7.65, a pH of from about 6.89 to about 6.93, and a mucoadhesive force (dyne/cm′÷SD) of from about 4.88 to about 6.0.

12. A composition for management of periodontal diseases, comprising:
a polymer system forming a gel matrix, the gel matrix including about 0.5% to about 1.5% by weight chitosan and about 20% to about 30% by weight poloxamer members;
a plurality of microspheres suspended and dispersed in the gel matrix;
an effective amount of an active ingredient for the management of periodontal disease, about one-half of the effective amount being dispersed in the gel matrix for rapid release of therapeutic levels of the active ingredient, the microspheres containing the remainder of the effective amount and providing gradual time release of the active ingredient to maintain the therapeutic levels of medicament for a sustained period of time.

13. The composition for management of periodontal diseases according to claim 12, wherein the poloxamer members include poloxamer 407 and poloxamer 188.

14. The composition for management of periodontal diseases according to claim 12, wherein said microspheres comprise a polymer selected from the group consisting of ethylcellulose, poly(lactide-co-glycolide) polymer, and polycaprolactone polymer.

15. The composition for management of periodontal diseases according to claim 12, wherein said active ingredient comprises an antibiotic.

16. The composition for management of periodontal diseases according to claim 12, wherein said active ingredient comprises ofloxacin.

17. The composition for management of periodontal diseases according to claim 12, wherein said active ingredient comprises an anesthetic.

18. The composition for management of periodontal diseases according to claim 12, wherein said active ingredient comprises mebeverine HCl.

19. The composition for management of periodontal diseases according to claim 12, wherein:
said gel matrix comprises about 15% to about 20% by weight poloxamer 407 and about 5% to about 15% by weight poloxamer 188;
said microspheres comprise a polymer selected from the group consisting of ethylcellulose, poly(lactide-co-glycolide) polymer, and polycaprolactone polymer; and
said active ingredient comprises about 0.05% by weight ofloxacin.

20. The composition for management of periodontal diseases according to claim 12, wherein:
said gel matrix comprises about 18% by weight poloxamer 407 and about 10% by weight poloxamer 188;
said microspheres comprise a polymer selected from the group consisting of ethylcellulose, poly(lactide-co-glycolide) polymer, and polycaprolactone polymer; and
said active ingredient comprises about 0.05% by weight ofloxacin.
